(19) United States
(12) Patent Application Publication
(10) Pub. No.: US 2014/0187817 A1
(43) Pub. Date: Jul. 3, 2014

(54) PREPARATION OF METHACRYLIC ACID

(71) Applicants: Torsten Balduf, Pfungstadt (DE); Martin Koestner, Darmstadt (DE); Wenxing Zhang, Darmstadt (DE); Heidi Groen, Frankfurt (DE); Axel Hengstermann, Senden (DE); Maya Krapfl, Singapore (SG)

(72) Inventors: Torsten Balduf, Pfungstadt (DE); Martin Koestner, Darmstadt (DE); Wenxing Zhang, Darmstadt (DE); Heidi Groen, Frankfurt (DE); Axel Hengstermann, Senden (DE); Maya Krapfl, Singapore (SG)

(73) Assignee: Evonik Roehm GmbH, Darmstadt (DE)

(21) Appl. No.: 14/240,547

(22) PCT Filed: Sep. 16, 2011
(86) PCT No.: PCT/CN2011/079765

§ 371 Date: Feb. 24, 2014

(51) Int. Cl. C07C 51/43

(52) U.S. Cl. CPC C07C 51/43
USPC 562/600

(57) ABSTRACT

The invention relates to a process for preparation of methacrylic acid, comprising process steps a) providing a crude methacrylic acid-comprising aqueous solution comprising at least one impurity at least partially dissolved therein; b) precipitation of at least a part of the at least one impurity from the crude methacrylic acid-comprising aqueous solution to form at least one solid impurity and a mother liquor; c) separation of at least a part of the at least one solid impurity from the mother liquor to obtain a purified methacrylic acid-comprising aqueous solution and a solid impurity; d) separation of methacrylic acid from the purified methacrylic acid-comprising aqueous solution.

The invention relates to a process for preparation of methacrylic acid.

Methacrylic acid (MAA) is used in a wide variety of applications. Methacrylic acid can be readily polymerized, its presence in copolymers reduces softening temperature and hardness and improves the adhesion of surface coatings and adhesives. Being a carboxylic acid, it can be converted by conventional methods into methacrylates, methacrylamide, N-substituted methacryl amides, and methacryloyl chloride. Polymers containing methacrylic acid are used in surface coatings, auxiliaries for the leather and textile industries, flocculants, ion exchangers, and soil improvers.

The commercial production of methacrylic acid occurs, among other ways, by heterogeneously catalysed gas phase oxidation of isobutylene, tert-butanol, methacrolein or isobutyl aldehyde. The thus obtained, gaseous reaction phase is transformed into an aqueous methacrylic acid solution by cooling and condensing, optionally separated from low-boiling substances such as, for example, acetaldehyde, acetone, acetic acid, acrolein and methacrolein and then introduced into a solvent extraction column, in order to extract and separate methacrylic acid by means of suitable extraction agents, such as, for example, short-chain hydrocarbons. The separated methacrylic acid is generally then further purified, for example by distillation, in order to obtain a pure methacrylic acid. Such a known process is described for example in EP 0 710 643, U.S. Pat. No. 4,618,709, US 4,956,493, EP 386 117 and U.S. Pat. No. 5,248,819.

The gaseous reaction phase generally also comprises various side-products, for example aromatic carboxylic acids such as terephthalic acid, benzoic acid, toluic acid and the like, maleic acid, citraconic acid, aldehydes, and polymers, among others. Such high-boiling products tend to solidify on cooling, which can lead to blocking of pipes and equipment, as well as increasing polymerisation of methacrylic acid and other products in the reaction mixture, which in turn leads to blocking, resulting in increased down times and decreased efficiency. These problems are not addressed in the above literature. Furthermore, some of these side-products are themselves of commercial interest, for example terephthalic acid (TPA) is a co-monomer in polyethylene terephthalate (PET) and also an important component in hybrid framework materials, used for storage, separation and purification of gases and in catalysis. Recovery of terephthalic acid is thus of interest. An object of the present invention is generally to overcome as far as possible the disadvantages of the prior art processes.

A further object is to increase the overall efficiency and/or yield of the methacrylic acid production process by reducing or eliminating blocking of pipes and equipment due to precipitation to the greatest extent possible.

A contribution to solving the above objects is made by a process for preparation of methacrylic acid, comprising process steps
a) providing a crude methacrylic acid-comprising aqueous solution comprising at least one impurity at least partially dissolved therein;
b) precipitation of at least a part of the at least one impurity from the crude methacrylic acid-comprising aqueous solution to form at least one solid impurity and a mother liquor;
c) separation of at least a part of the at least one solid impurity from the mother liquor to obtain a purified methacrylic acid-comprising aqueous solution and a solid impurity;
d) separation of methacrylic acid from the purified methacrylic acid-comprising aqueous solution.

In a preferred aspect of the process according to the invention process step a) comprises process steps:

a1) gas phase oxidation of a C₄ compound to obtain a methacrylic acid-comprising product gas;

a2) contacting the methacrylic acid-comprising product gas with a quenching agent to obtain a crude methacrylic acid-comprising aqueous solution comprising at least one impurity at least partially dissolved therein.

The C₄ compound which is subjected to gas phase oxidation in step al) of the process according to the invention is preferably a C₄ compound selected from isobutylene, tert-butyl alcohol, isobutylaldehyde and methacrolein, or a mixture of two or more thereof. In a preferred aspect of the invention, the C₄ compound is derived from splitting of methyl tert-butyl ether (MTBE) or ethyl tert-butyl ether (ETBE), and process step a) comprises further process step
aa1) splitting of MTBE to obtain at least one C₄ compound, preferably at least one of isobutylene and tert-butyl alcohol, and methanol.

MTBE is widely used as feedstock for isobutylene and splitting of MTBE is well known in the art. Splitting of MTBE can occur by any suitable means known to the skilled person. Suitable catalysts and reaction conditions are described, for example, in EP 1 149 814, WO 04/018393, WO 04/052809; Ullmann's Encyclopedia of Industrial Chemistry, 5^{th} Edition, Vol. A4, p. 488; V. Fattore, M. Massi Mauri, G. Oriani, G. Paret, Hydrocarbon Processing, August 1981, p. 101-106; Ullmann's Encyclopedia of Industrial Chemistry, 5^{th} Edition, Vol. A16, p. 543-550; A. Chauvel, G. Lefebvre, “Petrochemical Processes, Technical and Economic Characteristics”, Vol. 1, Editions Technip, Paris, 1989, p. 213 et seq.; U.S. Pat. No. 5,336,841, U.S. Pat. No. 4,570,026, and references cited therein. The disclosures of these references are hereby incorporated by reference and form part of the disclosure of the present invention.

The two main products of MTBE splitting are the C₄ compound isobutylene and methanol. The further C₄ compound tertiary-butanol can also be comprised in the splitting reaction product phase. Either or both of isobutylene and tertiary-butanol can be supplied as feed to process step a1), to make up the total C₄ compound content of the feed for this process step or in addition to further C₄ content from another source. One or more intermediate separation and/or purification steps are also possible between the splitting of MTBE and the supplying of the thus-obtained at least one C₄ compound to the gas phase oxidation in process step a1), for example, to separate as far as possible the at least one C₄ compound and methanol from each other and to remove any side products from the splitting which might adversely affect the gas phase oxidation. Separation and/or purification can be by any means known to the skilled person and appearing suitable. Suitable purification and separation processes are described, for example, in EP 1 149 814, WO 04/018393 and WO 04/052809. After separation of methanol, the splitting phase which comprises the C₄ compound isobutylene as main component can then be optionally purified and provided as feed to process step a1). Suitable purification methods are known to the person skilled in the art and preferably comprise at least one of distillation, extraction, adsorption, absorption, chromatography or washing, preferably at least one of distillation and extraction, preferably at least one distillation and at least one extraction. Unreacted MTBE can be at least partially separated from the C₄ compound phase in this step. Separated MTBE can be optionally purified and at least partially recycled to the splitting reaction.

The gas phase oxidation in step a1) of the process according to the invention preferably occurs in the presence of at least one oxidation catalyst. If the C₄ compound is isobutylene or tert-butyl alcohol, the gas phase oxidation to obtain a methacrylic acid-comprising gas phase can occur in one step, whereby one step in this context is considered to mean that initial oxidation to methacrolein and further oxidation to methacrylic acid occur substantially in the same reaction area, in the presence of at least one catalyst. Alternatively, the gas phase oxidation in step a1) can occur in more than one step, preferably in two steps, preferably in two or more reaction areas separated from each other, whereby two or more catalysts are preferably present, each catalyst preferably being present in a separate reaction area from each other catalyst. In a two-step gas phase oxidation, the first step is preferably at least partial oxidation of the C₄ compound to methacrolein, followed by at least partial oxidation of methacrolein to methacrylic acid. Accordingly, for example, in a first reaction step, preferably at least one catalyst suitable for oxidation of at least one C₄ compound to methacrolein is present, and in a second reaction step, at least one catalyst suitable for oxidation of methacrolein to methacrylic acid is present.

Suitable reaction conditions for gas phase catalytic oxidation are, for example, temperatures of from about 250° C. to about 450° C., preferably from about 250° C. to about 390° C. and pressures of from about 1 atm. to about 5 atm. The space velocity can vary from about 100 to about 6000 per hr (NTP) and preferably from about 500 to about 3000 per hr. Oxidation, for example gas phase catalytic oxidation, of C₄ feeds such as isobutylene to methacrolein and/or methacrylic acid, as well as catalysts therefor, are well known in the literature, for example from U.S. Pat. No. 5,248,819, U.S. Pat. No. 5,231,226, U.S. Pat. No. 5,276,178, U.S. Pat. No. 6,596,901, U.S. Pat. No. 4,652,673, U.S. Pat. No. 6,498,270, U.S. Pat. No. 5,198,579, U.S. Pat. No. 5,583,084.

Particularly preferred catalysts and processes suitable for oxidation of isobutylene or tert-butanol to methacrolein and/or methacrylic acid are described in EP 0 267 556, and particularly preferred catalysts and processes suitable for oxidation of methacrolein to methacrylic acid are described in EP 0 376 117. These documents are hereby introduced as reference and form part of the disclosure of the present invention.

The gas phase oxidation of methacrolein to methacrylic acid in the process according to the invention preferably occurs at temperatures of from about 250 to about 350° C. and below, at pressures from about 1 to about 3 atm, and at volume loads of from about 800 to about 1800 NI/l/h.

As oxidising agent, generally oxygen is used, for example, in the form of air, or in the form of pure oxygen or oxygen diluted with at least one gas which is inert under the reaction conditions, such as at least one of nitrogen or carbon dioxide, whereby air is preferred as oxidising agent and nitrogen and/or carbon dioxide are preferred as diluent gas. If carbon dioxide is used as diluent gas, this is preferably carbon dioxide recycled from a combustion, preferably a catalytic or thermal combustion of reaction gases and/or byproducts. The gas subjected to gas phase oxidation in step al) of the process according to the invention preferably also comprises water, which is generally present in the form of water vapour. The oxygen, inert gas or gases and water can be introduced into the reaction phase or combined with the C₄ compound before or during or before and during the gas phase reaction.

In a preferred embodiment of the process according to the invention, a mixture comprising at least one C₄ compound, air or oxygen and recycled oxidation reactor exit gas, preferably oxidation reactor exit gas which has been combusted prior to recycling, is supplied to step al). The reactor exit gas preferably comprises at least one unreacted C₄ compound, at least one carbon oxide, nitrogen and oxygen, as well as water, depending on the separation conditions and the presence of and action of a combustion step.

In a two-step gas phase oxidation according to the invention, a preferred volume ratio in the first step of C₄ compound:O₂ :H₂O:inert gas is generally 1:0.5-5:1-20:3-30, preferably 1:1-3:2-10:7-20. The volume ratio in the second step of methacrolein:O₂ :H₂O:inert gas is preferably 1:1-5:2-20:3-30, preferably 1:1-4:3-10:7-18.

In step a2) of the process according to the invention, the gas phase which comprises methacrylic acid is cooled and condensed by contacting with a quenching agent—commonly known as quenching—to obtain a condensate in the form of a crude aqueous methacrylic acid-comprising solution. The cooling and condensation can occur by any means known to the skilled person and appearing suitable, for example by cooling the methacrylic acid-comprising gas phase to temperatures below the dew point of at least one of its components, in particular of at least one of water and methacrylic acid. Suitable methods of cooling are known to the skilled person, for example, cooling by means of at least one heat exchanger, and/or by spraying the gas phase with a liquid, for example with water, an aqueous composition or an organic solvent, such as, for example, an organic solvent selected from aromatic or aliphatic hydrocarbons, or a mixture of at least two thereof, whereby preferred organic solvents have relatively low vapour pressure under the quenching conditions, such as heptane, toluene or xylene, whereby water is preferred as quench liquid according to the invention, and at least a portion of the condensate formed in the quenching step itself is even more preferred. Suitable quenching processes are known to the skilled person, for example from DE 21 36 396, EP 297 445, EP 297 788, JP 01193240, JP 01242547, JP 01006233, US 2001/0007043, U.S. Pat. No. 6,596,901, U.S. Pat. No. 4,956,493, U.S. Pat. No. 4,618,709, U.S. Pat. No. 5,248,819, whose disclosure concerning quenching of acrylic and methacrylic acids is hereby incorporated and forms part of the present disclosure. It is preferred according to the invention that the gas phase is cooled to temperatures between 40 and 80° C. and washed with water and/or condensate from the quenching step to obtain an aqueous solution comprising methacrylic acid, which can also comprise varying amounts of impurities such as acetic acid, maleic acid, fumaric acid, citraconic acid, acrylic acid and formic acid, as well as aromatic acids such as benzoic acid, toluic acid and terephthalic acid, and aldehydes such as formaldehyde, acetaldehyde, propionaldehyde, acrolein, methacrolein, ketones and unreacted C₄ compound or compounds. These impurities, as well as water, need to be separated to the greatest extent possible from the methacrylic acid in order to obtain a high degree of purity of methacrylic acid. While it is possible to separate some of the impurities by means of thermal separation processes such as distillation, or by washing, extraction and the like, these types of separation are not suitable for separating all of the impurities, in particular those which are only poorly soluble in solvents such as water, and thus readily precipitate from solutions, for example terephthalic acid. For such impurities, other separation means, such as those described herein, are more effective.

The crude methacrylic acid-comprising aqueous solution exiting process step a2) generally has a temperature in the range from about 65° C. to about 80° C. This crude methacrylic acid-comprising solution is preferably cooled in a first cooling step to a temperature in the range of from about 0 to about 75° C., preferably in the range of from about 5 to about 65° C., more preferably in the range of from about 10 to about 60° C., yet more preferably in the range of from about 15 to about 55° C., even more preferably in the range of from about 20 to about 45° C., in order to facilitate and accelerate precipitation in step b) of the process according to the invention. Lower temperatures in the preferred ranges are preferred, since they favour precipitation of the at least one impurity whereby temperatures should be selected which do not also lead to precipitation of methacrylic acid. Such a first cooling of the crude methacrylic acid-comprising solution can take place in an intermediate step following the quenching of step a2) and prior to step b) of the process according to the invention, or at entry into process step b), or during process step b), or any combination thereof.

In step b) of the process according to the invention at least a part, preferably at least 50 wt. %, preferably at least 60 wt. %, more preferably at least 70 wt. %, yet more preferably at least 80 wt. %, even more preferably at least 90 wt. %, more preferably at least 95 wt. %, respectively based on the amount of the respective at least one impurity present in the crude methacrylic acid-comprising aqueous solution, of the at least one impurity is precipitated from the crude methacrylic acid-comprising aqueous solution to form at least one solid impurity. The terms “precipitate”, “precipitation” and the like are intended to mean any of precipitate, crystal or crystals, precipitation and crystallisation, as well as any other transfer of a dissolved material into an undissolved, solid state. The at least one impurity which is precipitated can be any one or more of the impurities comprised in the crude methacrylic acid-comprising solution, and is preferably at least terephthalic acid.

The precipitation in step b) of the process according to the invention can occur in one precipitation stage or in two or more precipitation stages, whereby a two-stage precipitation generally results in better separation of at least one impurity from the crude methacrylic acid-comprising solution and is thus preferred according to the invention. The crude methacrylic acid-comprising aqueous solution is preferably introduced into a first precipitation area, where at least one impurity precipitates in the form of a solid or crystals. In a one-stage precipitation, the precipitate is then separated at least partially from the mother liquor in step c) of the process according to the invention by any suitable solid-liquid separation means, such as filtration, centrifugation or the like. In a two-stage precipitation, the precipitate from the first precipitation area, together with the mother liquor, is conducted in the form of a suspension or a slurry to a second precipitation area, where further precipitation, crystallisation and/or crystal growth occurs. The precipitate from the second precipitation area is then separated at least partially from the mother liquor in step c) of the process according to the invention by any solid-liquid separation means known to the skilled person and appearing suitable, such as filtration, centrifugation or the like. The duration of the respective precipitation stages is preferably controlled by the fill level of the respective area, whereby once a given fill level has been reached at least a part of the slurry of precipitate and mother liquor is conducted to at least one of the next precipitation area and the next process step. While this can occur continuously or discontinuously, in a particularly preferred aspect of the process according to the invention, this occurs continuously. As a guide for preferred duration times for the precipitation stages, in a two-stage precipitation, the first precipitation stage preferably has a duration in the range of from about 1 to about 36 hours, preferably in the range of from about 2 to about 30 hours, more preferably in the range of from about 3 to about 25 hours, more preferably in the range of from about 4 to about 20 hours, yet more preferably in the range of about 3 to about 8 hours, while the second precipitation stage preferably has a duration in the range of from about 1 to about 24 hours, preferably in the range of from about 1 to about 20 hours, more preferably in the range of from about 1.5 to about 15 hours, more preferably in the range of from about 2 to about 10 hours, even more preferably in the range of from about 2 to about 5 hours.

It is preferred in the process according to the invention that in step b) the precipitation is at least partially carried out with agitation of the crude methacrylic acid-comprising aqueous solution. In a two-stage precipitation agitation can occur in one or both stages and preferably occurs in both stages. Agitation of the crude methacrylic acid-comprising solution assists precipitation on the one hand by bringing already precipitated solid matter to the evaporation surface, and on the other hand by bringing already precipitated solid matter into contact with more highly saturated parts of the solution, both of which result in increased precipitation and increased particle size of the precipitated solid matter.

In a preferred aspect of the process according to the invention, in process step b) at least a part, preferably more than 30 wt. %, preferably at least 40 wt. %, more preferably at least 50 wt. %, even more preferably at least 60 wt. %, even more preferably at least 70 wt. %, yet more preferably at least 80 wt. %, more preferably at least 90 wt. %, even more preferably all of the crude methacrylic acid-comprising aqueous solution, based on the total weight of the crude methacrylic acid-comprising aqueous solution exiting process step a2), is introduced into a first precipitation area. Precipitation occurs in the first precipitation area, corresponding to the first precipitation stage of a two-stage precipitation in process step b). The dwell time of the crude methacrylic acid-comprising solution in the first precipitation area is preferably controlled by means of the fill level, as described above, preferably in such a way as to allow a continuous process, whereby as a guide the crude methacrylic acid-comprising solution preferably has a dwell time in the first precipitation area in the range of from about 1 to about 36 hours, preferably in the range of from about 2 to about 30 hours, more preferably in the range of from about 3 to about 25 hours, more preferably in the range of from about 4 to about 20 hours, yet more preferably in the range of from about 3 to about 8 hours, during which the solution is preferably agitated while precipitation occurs. A cooling of the crude methacrylic acid-comprising solution can take place in the first precipitation area, or at or prior to its entry into the first precipitation area, or any combination thereof. An advantage of cooling prior to or at entry into the first precipitation area is that a more efficient cooling of the solution can be achieved as it flows through a cooling device, for example a heat exchanger, compared to cooling a larger volume of the solution after entry into the first precipitation area. However, a cooling considerably before entry into the cooling device could lead to premature precipitation of at least one impurity, which could in turn result in blocked pipes and reduced overall efficiency of the process. Cooling is thus preferably carried out as close as possible to the point of entry into the first precipitation area, or in the first precipitation area, or both.

It is preferred in the process according to the invention that in step b) a precipitation seed is provided to the crude methacrylic acid-comprising aqueous solution. The precipitation seed can be any material known to the skilled person and appearing suitable for promoting precipitation of at least one impurity, for example a finely divided solid, preferably a crystalline solid, such as at least one of a crystallisation aid, a filtration aid, and at least one of the impurities to be separated, in the form of crystals or fine solid material. At least one of the impurities to be separated is preferred as precipitation seed, whereby terephthalic acid is particularly preferred and terephthalic acid exiting and recycled from the first precipitation area or from a further precipitation area is most preferred. In a particularly preferred aspect of the process according to the invention, terephthalic acid in the form of a flow of precipitate and mother liquor leaving at least one of the first precipitation area and the second precipitation area is conducted back to the crude methacrylic acid-comprising aqueous solution in the first precipitation area. If the precipitation seed is conducted from the second precipitation area to the first precipitation area, it is preferred that this is in the form of a flow of fine precipitate drawn off through a side outlet of the second precipitation area, preferably at a side outlet at an upper level of the second precipitation area, preferably at a level in the top half of the second precipitation area.

According to a preferred aspect of the process according to the invention, at least 50 wt. % of the precipitation seed has a particle size in the range of from 1 to 200 μm, preferably from 1 to 100 μm, more preferably from 1 to 50 μm, yet more preferably from 1 to 25 μm, measured by a method described herein. Particle size is measured according to ISO 13320-1:1999(E): “Particle Size Analysis—Laser Diffraction Methods”

It is preferred in the process according to the invention that the providing of the precipitation seed to the crude methacrylic acid-comprising aqueous solution occurs with agitation to form a precipitation mixture. This agitation, preferably in the form of stirring, has the same advantages as mentioned above in connection with the agitation of the crude methacrylic acid-comprising solution during precipitation. The thus-formed precipitation mixture is thus a mixture comprising precipitation seed and crude methacrylic acid-comprising aqueous solution, as well as any precipitate from the crude methacrylic acid-comprising aqueous solution. The agitation preferably leads to a substantially uniform distribution of the solid matter throughout the precipitation mixture.

It is preferred in the process according to the invention that the precipitation seed is at least partially pre-formed in a further precipitation area, preferably in a second precipitation area, before being provided to the crude methacrylic acid-comprising aqueous solution. In this aspect, the second precipitation area can correspond to the second precipitation stage as described above, where the precipitation seed is at least one impurity which is to be, or has been, precipitated from the crude methacrylic acid-comprising aqueous solution.

In this aspect of the process according to the invention, the precipitation seed is preferably provided to the crude methacrylic acid-comprising aqueous solution via a side outlet at the second precipitation area. Preferably, a flow of fine precipitate in the form of a suspension or slurry in mother liquor is drawn off through a side outlet at an upper level of the second precipitation area, preferably at a level in the top half of the second precipitation area. This is then preferably provided to the crude methacrylic acid-comprising aqueous solution in the first precipitation area.

In a preferred aspect of the process according to the invention, the precipitation seed is provided to the crude methacrylic acid-comprising aqueous solution in the first precipitation area, the first precipitation area corresponding to the first precipitation stage. In this way a precipitation of at least one impurity in the first precipitation stage should be encouraged and accelerated, thereby reducing the residence time necessary to achieve a desired amount of precipitation and/or a desired particle size of the precipitate.

Preferably, in the process according to the invention, at least a part of the precipitation mixture is conducted to the second precipitation area. The precipitation mixture conducted to the second precipitation area is then preferably agitated in the second precipitation area while further precipitation and/or crystal growth occurs.

In this aspect of the process according to the invention, the at least a part of the precipitation mixture is conducted to the second precipitation area via a side or bottom outlet, preferably via a side outlet, at the first precipitation area. Conducting via a side outlet of the first precipitation area is preferred in particular if the agitation in the first precipitation area is not carried out continuously, so that, for example, during periods where no agitation is carried out, larger solid particles of precipitate, such as larger crystals, are allowed to settle towards a lower region of the first precipitation area, while finer particles remain suspended in the mother liquor for longer and can thus be decanted off at a higher level of the first precipitation area compared to the larger particles. In principle, the side outlet can be at any level of the first precipitation area, provided that this level is no higher than the maximum fill level of the first precipitation area.

In a further preferred aspect of the process according to the invention, in step b) a further cooling occurs, preferably a cooling of the precipitation mixture in the second precipitation area. This further cooling is preferably to a temperature in the range of from about 0° C. to about 20° C., preferably in the range of from about 5° C. to about 15° C. The cooling is preferably to a temperature at which the precipitation of at least one impurity is encouraged and accelerated, without at the same time causing precipitation of methacrylic acid.

In step c) of the process according to the invention, at least a part of the at least one solid impurity is separated from the mother liquor to obtain a purified methacrylic acid-comprising aqueous solution and a solid impurity.

It is preferred in step c) of the process according to the invention that a stream comprising at least a part of the solid impurity exits the second precipitation area via an outlet arranged in a lower region of the second precipitation area and is conducted to a separation unit. The stream comprising at least a part of the solid impurity is preferably a stream comprising solid impurity and mother liquor in the form of a suspension or slurry. The separation unit can comprise one or more separation areas. If more than one separation area is comprised, the stream can be conducted first to one separation area and, only when this separation area is filled, conducted to at least one further separation area, or the stream can be conducted substantially simultaneously to two or more separation areas. The separation in process step c) can occur by any solid-liquid separation means known to the skilled person and appearing suitable, whereby filtration and centrifugation are preferred and filtration is particularly preferred. The separated solid impurity is preferably collected and optionally washed and/or purified, if it is to be conducted to further applications, treatment or derivatisation. The separated mother liquor forms the purified methacrylic acid-comprising aqueous solution and is further treated in further process steps in order to obtain at least a part of the methacrylic acid comprised therein.

The separation of methacrylic acid from the purified methacrylic acid-comprising aqueous solution in step d) of the process according to the invention preferably occurs by means of extraction into an organic extraction agent. Preferred organic extraction agents are, for example, at least one organic solvent, preferably at least one organic solvent which is substantially immiscible with water, such that an aqueous phase and an organic phase can be formed. Process step d) also comprises the separation of the aqueous and organic phases from each other. Preferred organic solvents which can be used in step d) of the process according to the invention have a boiling point different to, preferably lower than, the boiling point of methacrylic acid. Preferably, in the process according to the invention, the organic extraction agent used in process step d) has a boiling point of less than 161° C. measured at atmospheric pressure. The organic extraction agent can then in principle be separated from methacrylic acid in a further process step, for example by distillation, where it is preferably at least partially removed as a low boiler at a higher level in the distillation apparatus than the separated methacrylic acid. The separated organic extraction agent or a part thereof can be conducted back to process step d), optionally after at least one cooling and/or purification step. Preferred organic solvents for this step are in particular selected from alkanes and aromatic, preferably alkylaromatic, hydrocarbons, whereby at least one organic solvent selected from a C₆-C₈ hydrocarbon is preferred, whereby heptane, toluene and xylene are particularly preferred and heptane, preferably n-heptane is most preferred. Process step d) can be carried out by any means known and appearing suitable to the skilled person, preferably as a countercurrent extraction, for example by means of a solvent extraction column, a pulsed fill or packing column, rotating extractors, a washing column, a phase separator or other device suitable for extraction of an aqueous phase with an organic solvent and separation of the organic phase from the aqueous phase. It is preferred according to the invention that at least a part, preferably at least 50 wt. %, preferably at least about 70 wt. %, preferably at least about 80 wt. %, more preferably at least about 90 wt. % of the methacrylic acid comprised in the aqueous methacrylic acid solution is extracted into the organic extraction agent. Two phases are thus obtained in step d) of the process according to the invention: an organic phase comprising methacrylic acid and extraction agent, and an aqueous phase which is generally considered as waste water. The organic phase is preferably subjected to a separation, preferably a thermal separation process to separate at least a part of the methacrylic acid comprised therein from the organic extraction agent. If a thermal separation is used, this is preferably a distillation, whereby the extraction agent is preferably removed as head product or at an upper level of a distillation column, while methacrylic acid or a methacrylic acid-rich phase is removed as bottom product or at a lower level of the distillation column than the extraction solvent. It is also possible to use, for example, a fractionating or rectification column, so that impurities with boiling points higher than methacrylic acid remain in the bottom product and methacrylic acid of higher purity can be removed at a level which is higher than the bottom of the column. If the organic solvent used for extraction has a higher boiling point than the boiling point of methacrylic acid, it is also possible to remove methacrylic acid phases at the top and/or higher levels of the column.

The invention also relates to terephthalic acid obtainable by the process according to the invention.

The invention is more closely illustrated by the following figures and non-limiting examples.

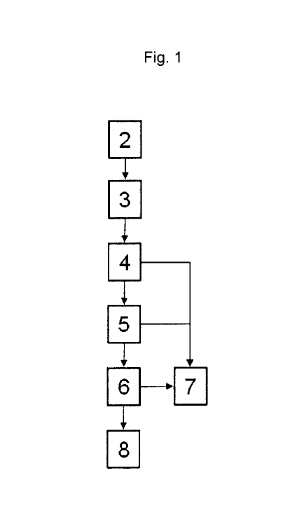

The embodiment of the inventive process as illustrated in FIG. 1 shows the arrangement of the process components with respect to each other. The crude methacrylic acid-comprising solution is conducted from supply 2 to precipitation unit 3, where at least a part of at least one impurity precipitates to form a precipitate and a mother liquor. The mixture of precipitate and mother liquor is conducted to first separation unit 4 where precipitate and mother liquor are separated from each other. The precipitate, separated in solid form, can, if desired, be washed, and can be conducted to further process steps such as derivatisation or polycondensation (not shown) or reaction with further components. This can be particularly preferred if the precipitate comprises terephthalic acid. The mother liquor is conducted to a second separation unit 5, where methacrylic acid is extracted from the aqueous mother liquor into an organic solvent. Alternatively or in addition thereto, a part or all of the mother liquor can be conducted to an esterification unit 7 for esterification of the methacrylic acid comprised therein or to a storage unit 8. A part or all of the organic phase comprising methacrylic acid which exits second separation unit 5 can be conducted to purification unit 6, or to the esterification unit 7. In purification unit 6, at least a part of the methacrylic acid is separated from the organic extraction agent and optionally further purified. The thus-obtained methacrylic acid can be removed, or conducted to the esterification unit 7. In esterification unit 7, methacrylic acid is esterified with an alcohol to form the corresponding methacrylic acid ester in an ester phase.

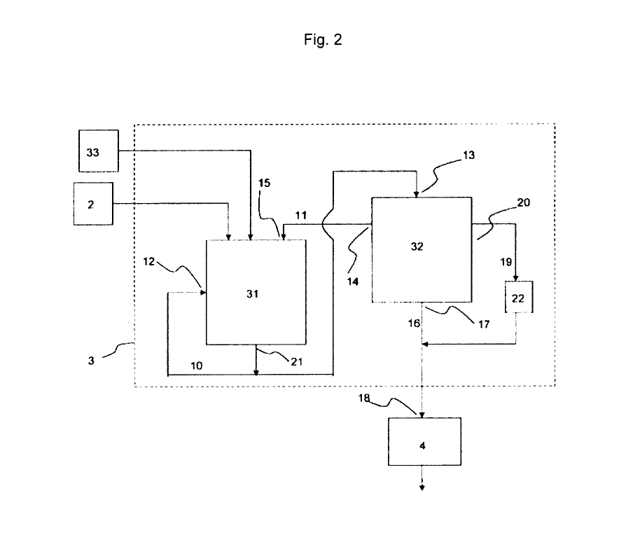

FIG. 2 shows a preferred embodiment of the precipitation unit 3 of the process according to the invention inside the dashed line, with further process components shown outside the dashed line. From supply 2, optionally via a cooling unit (not shown) a crude methacrylic acid-comprising solution is conducted to residence container 31. Optionally, a crystallisation aid or filtration aid is also conducted to residence container 31 from reservoir 33. A precipitation seed, for example in the form of a suspension or slurry of fine precipitate, is optionally conducted to residence container 31 from crystalliser 32 via line 11, exiting crystalliser 32 at outlet 14 and entering residence container 31 at inlet 15.

The contents of residence container 31 can be agitated by means of an external or internal agitator (not shown) in order to combine the contents. Cooling or heating can also take place. When a certain amount of precipitation has occurred, and/or a certain fill level is reached inside the residence container 31, at least a part of the contents of the residence container is conducted by means of line 10, either via outlet 12 or via outlet 21, to crystalliser 32, entering at inlet 13. This preferably occurs continuously. The contents of crystalliser 32 are agitated by means of an internal agitator (not shown) and preferably also cooled. The agitation is preferably such that larger precipitate particles are directed to the bottom of crystalliser 32, where they can be removed via outlet 17 and conducted via line 16 to first separation unit 4, entering at inlet 18. The fine precipitate particles preferably remain in crystalliser 32 so that they can grow in size, whereby at least a part thereof can be conducted via line 11 to residence container 31 as precipitation seed. If the separation in separation unit 4 is slow relative to the rate of entry of the feed from crystalliser 32, resulting in a capacity bottleneck at separation unit 4, at least a part of the contents of crystalliser 32 can be drawn off at side outlet 20 and conducted via line 19 to buffer tank 22 until the bottleneck is removed or dissipates. The separation in separation unit 4, as well as other device components and processes carried out therein, are as described for FIG. 2.

### EXAMPLE 1

2.5 L/h of a crude MAA Solution (37% MAA) with a TPA content of 600 ppm is fed to a stirred tank with a volume of 8 L. The stirred tank is cooled to 13° C. The stirred tank has an external pump with a capacity of around of 40 L/h. 27.5 L/h are recycled back to the first stirred tank and 12.5 L/h are fed to a crystallizer (Volume 8 L) with slow agitation. This container is also externally cooled to 13° C. At the top of the crystallizer 10 L/h are withdrawn and fed back to the first stirred tank. This flow is adjusted manually to remove fine particles with a particle size less than 25 μm from the crystallizer. These fine particles are the seed crystals for the first stirred tank. At the bottom of the second stirred tank 2.5 L/h are removed and filtered in a filter funnel. The bottom product contains 110 ppm TPA.

Particle size is measured according to ISO 13320-1:1999(E): “Particle Size Analysis—Laser Diffraction Methods”

1. A process for preparing methacrylic acid, the process comprising
a) precipitating, from a crude methacrylic acid-comprising aqueous solution comprising an impurity at least partially dissolved therein, at least a part of the impurity to form at least one solid impurity and a mother liquor;
b) separating at least a part of the at least one solid impurity from the mother liquor to obtain a purified methacrylic acid-comprising aqueous solution and a solid impurity; and
c) separating methacrylic acid from the purified methacrylic acid-comprising aqueous solution.

2. The process according to claim 1, wherein the crude methacrylic acid-comprising aqueous solution is prepared by a process comprising:
(i) oxidizing a C₄ compound in a gas phase to obtain a methacrylic acid-comprising product gas; and
(ii) contacting the methacrylic acid-comprising product gas with a quenching agent to obtain the crude methacrylic acid-comprising aqueous solution.

3. The process according to claim 1, wherein in said precipitating a), the crude methacrylic acid-comprising aqueous solution is provided at a temperature in a range of from 10 to less than 55° C.

4. The process according to claim 1, wherein said precipitating a) is at least partially carried out with agitation of the crude methacrylic acid-comprising aqueous solution.

5. The process according to claim 1, wherein in said precipitating a), at least a part of the crude methacrylic acid-comprising aqueous solution is introduced into a first precipitation area.

6. The process according to claim 1, wherein in said precipitating a), a precipitation seed is provided to the crude methacrylic acid-comprising aqueous solution.

7. The process according to claim 6, wherein at least 50 wt. % of the precipitation seed has a particle size of from 1 to 200 μm, measured according to ISO 13320-1:1999(E).

8. The process according to claim 6, wherein the precipitation seed is provided with agitation to form a precipitation mixture.

9. The process according to claim 6, wherein the precipitation seed is at least partially pre-formed in a second precipitation area before being provided to the crude methacrylic acid-comprising aqueous solution.

10. The process according to claim 9, wherein the precipitation seed is provided to the crude methacrylic acid-comprising aqueous solution via a side outlet at the second precipitation area.

11. The process according to claim 6, wherein the precipitation seed is provided to the crude methacrylic acid-comprising aqueous solution in a first precipitation area.

12. The process according to claim 8, wherein at least a part of the precipitation mixture is conducted to a second precipitation area.

13. The process according to claim 12, wherein the at least a part of the precipitation mixture is conducted to the second precipitation area via a side outlet at a first precipitation area.

14. The process according to claim 1, wherein said precipitating a) further comprises cooling.

15. The process according to claim 1, wherein in said separating b), a stream comprising at least a part of the solid impurity exits a second precipitation area via an outlet arranged in a lower region of the second precipitation area and is conducted to a separation area.
